(19) United States
(12) Patent Application Publication
(10) Pub. No.: US 2023/0066856 A1
(43) Pub. Date: Mar. 2, 2023

(54) Dry Electrode And Wearable Device

(71) Applicant: TERUMO KABUSHIKI KAISHA, Tokyo (JP)

(72) Inventor: Masaomi IMAI, Chuo (JP)

(73) Assignee: TERUMO KABUSHIKI KAISHA, Tokyo (JP)

(21) Appl. No.: 17/821,912

(22) Filed: Aug. 24, 2022

(30) Foreign Application Priority Data
Aug. 26, 2021 (JP) 2021-138180

(51) Int. Cl. A61B 5/251; A61B 5/268

(52) U.S. Cl. CPC A61B 5/251; A61B 5/268; A61B 2562/14; A61B 2562/0209

(57) ABSTRACT

A dry electrode and a wearable device are configured to improve the adhesiveness with a skin. The dry electrode includes: an electrode core; and an electrode pad that is conducted to the electrode core, in which the electrode core includes a plate portion that is surrounded by the electrode pad, and a core main body portion that protrudes from the plate portion, and is at least partially exposed to an outside of the electrode pad, and the electrode pad includes conductive rubber or a conductive elastomer having a volume resistivity of 0.5 to 10.0 Ω·cm.

### CROSS-REFERENCE TO RELATED APPLICATION

This application is based on and claims priority to Japanese Patent Application No. 2021-138180 filed on Aug. 26, 2021, the entire content of which is incorporated herein by reference.

### TECHNOLOGICAL FIELD

This disclosure generally relates to a dry electrode and a wearable device.

### BACKGROUND DISCUSSION

The bioelectricity measurement of an individual depends on an electric parameter of an interface between each electrode and the individual's skin. Examples of the bioelectricity measurement include an electrocardiogram (ECG, EKG), an electroencephalogram (EEG), an electromyogram (EMG), a galvanic skin response, and a biological impedance. In the differential measurement, some sort of mismatch of an impedance between the electrode and the skin may cause shifts of a phase and an amplitude that degrade the degrees of fidelity for the measurement and the diagnosis, in measurement signals. An impedance between the skin and the electrode is measured from the skin as the maximum impedance element. The skin has an impedance that largely varies, and that is not directly measured with a some sort of accuracy in some cases. Moreover, the skin impedance varies depending on time, a subject, and an environment.

Japanese Patent Application Publication No. 2018-153357 discloses a sensing device including a metal electrode that is adhered to a skin of a user with a conductive paste therebetween.

In the bioelectricity measurement, generally, a wet electrode such as a hydrogel electrode is used. The wet electrode is attached to a skin with a gel-type adhesive compound after the skin is wiped in advance with alcohol or the like. The wet electrode uses the gel-type adhesive compound, thereby optimizing the impedance between the skin and the electrode, and providing the good signal quality.

Meanwhile, the wet electrode that uses the gel-type adhesive compound gives a subject unpleasant feeling in some cases. Moreover, the gel-type adhesive compound in the wet electrode dries over time. Accordingly, when the wet electrode is used for long periods, the adhesiveness with the skin varies due to the dry, and the accuracy of a measurement value may be lowered. In order to prevent the lowering in the accuracy of the measurement value, the wet electrode needs to be frequently replaced, and the work is also cumbersome.

### SUMMARY

Disclosed here are a dry electrode and a wearable device capable of improving the adhesiveness with a skin.

A dry electrode as a first aspect of this disclosure includes: an electrode core; and an electrode pad that is conducted to the electrode core, in which the electrode core includes a plate portion that is surrounded by the electrode pad, and a core main body portion that protrudes from the plate portion, and is at least partially exposed to an outside of the electrode pad, and the electrode pad includes conductive rubber or a conductive elastomer having a volume resistivity of 0.5 to 10.0 Ω·cm.

As one embodiment in this disclosure, the Shore A hardness of the electrode pad is 50 to 80.

As one embodiment in this disclosure, an outer surface of the electrode pad includes a contact surface that comes into contact with a surface of a living body, and the contact surface includes a protruding surface.

As one embodiment in this disclosure, the plate portion of the electrode core has an oval or circular outer shape in a plan view seen from a thickness direction.

As one embodiment in this disclosure, the core main body portion of the electrode core includes a through hole sectioned at a position where the core main body portion is exposed from the electrode pad.

As one embodiment in this disclosure, an outer surface of the core main body portion of the electrode core includes a recessed portion at a position where the core main body portion is exposed from the electrode pad.

As one embodiment in this disclosure, the electrode core includes at least one among gold, a gold alloy, silver, a silver alloy, copper, a copper alloy, stainless steel, and carbon.

As one embodiment in this disclosure, at least the outer surface of the electrode core includes gold.

A wearable device as a second aspect in this disclosure includes a dry electrode and a device main body in which the dry electrode is held. The dry electrode comprises an electrode core and an electrode pad electrically conductive with the electrode core, with the electrode core including a plate portion surrounded by the electrode pad. A core main body portion protrudes away from the plate portion and is at least partially exposed to an outside of the electrode pad. The electrode pad is comprised of conductive rubber or a conductive elastomer having a volume resistivity of 0.5 to 10.0 Ω·cm. The device main body includes a mounting portion configured to be mounted on a living body, and the dry electrode being held in the device main body with the electrode pad of the dry electrode exposed so that when the mounting portion of the device main body is mounted on the living body the electrode pad of the dry electrode is brought into contact with a surface of the living body.

With this disclosure, it is possible to provide a dry electrode and a wearable device capable of improving the adhesiveness with a skin.

In accordance with another aspect, a method comprises positioning a wearable device on a living body, wherein the wearable device comprises a device main body in which is held a dry electrode, with the dry electrode comprising an electrode core and an electrode pad electrically conductive with the electrode core. The electrode core includes a plate portion and a core main body portion protruding away from the plate portion, with the plate portion of the electrode core being surrounded by the electrode pad, at least a part of the core main body portion that protrudes away from the plate portion being exposed outside the electrode pad. The electrode pad is comprised of conductive rubber or a conductive elastomer having a volume resistivity of 0.5 to 10.0 Ω·cm. The positioning of the wearable device on the living body comprises positioning the wearable device so that an exposed surface of the electrode pad of the dry electrode directly contacts a surface of the living body

### BRIEF DESCRIPTION OF THE DRAWINGS

FIG. 1 is a perspective view illustrating a dry electrode as a first embodiment in this disclosure.

FIG. 2 is a top view of the dry electrode illustrated in FIG. 1.;

FIG. 3 is a perspective view illustrating a simple piece of an electrode core of the dry electrode illustrated in FIG. 1.

FIG. 4A is a cross-sectional view of the dry electrode at a position along the section line 4A-4A in FIG. 2.

FIG. 4B is a cross-sectional view of the dry electrode at a position along the section line 4B-4B in FIG. 2.

FIG. 5 is a perspective view illustrating a dry electrode as a second embodiment in this disclosure.

FIG. 6 is a top view of the dry electrode illustrated in FIG. 5.

FIG. 7A is a cross-sectional view of the dry electrode at a position along the section line 7A-7A in FIG. 6.

FIG. 7B is a cross-sectional view of the dry electrode at a position along the section line 7B-7B in FIG. 6.

FIG. 8 is a perspective view illustrating a dry electrode as a third embodiment in this disclosure.

FIG. 9 is a top view of the dry electrode illustrated in FIG. 8.

FIG. 10A is a cross-sectional view of the dry electrode at a position along the section line 10A-10A in FIG. 9.

FIG. 10B is a cross-sectional view of the dry electrode at a position along the section line 10B-10B in FIG. 9.

FIG. 11 is a perspective view illustrating a dry electrode as a fourth embodiment in this disclosure.

FIG. 12 is a top view of the dry electrode illustrated in FIG. 11.

FIG. 13A is a cross-sectional view of the dry electrode at a position along the section line 13A-13A in FIG. 12.

FIG. 13B is a cross-sectional view of the dry electrode at a position along the section line 13B-13B in FIG. 12.

FIG. 14A is a top view illustrating a dry electrode as a fifth embodiment in this disclosure.

FIG. 14B is a side view of the dry electrode illustrated in FIG. 14A.

FIG. 15A is a perspective view of a dry electrode array in which the dry electrodes illustrated in FIG. 1 are interlocked.

FIG. 15B is a top view of the dry electrode array illustrated in FIG. 15A.

FIG. 16 is a perspective view of a wearable device as one embodiment in this disclosure.

FIG. 17 is a use state view illustrating a state where the wearable device illustrated in FIG. 16 is mounted on a wrist of a subject so as to cover his/her skin.

FIG. 18 is a cross-sectional view along the section line 18-18 in FIG. 17.

### DETAILED DESCRIPTION

Set forth below with reference to the accompanying drawings is a detailed description of embodiments of a dry electrode and a wearable device representing examples of the new dry electrode and wearable device disclosed here. Features of the dry electrodes and wearable devices that are common amongst several embodiments are identified by the same reference numerals.

### First Embodiment

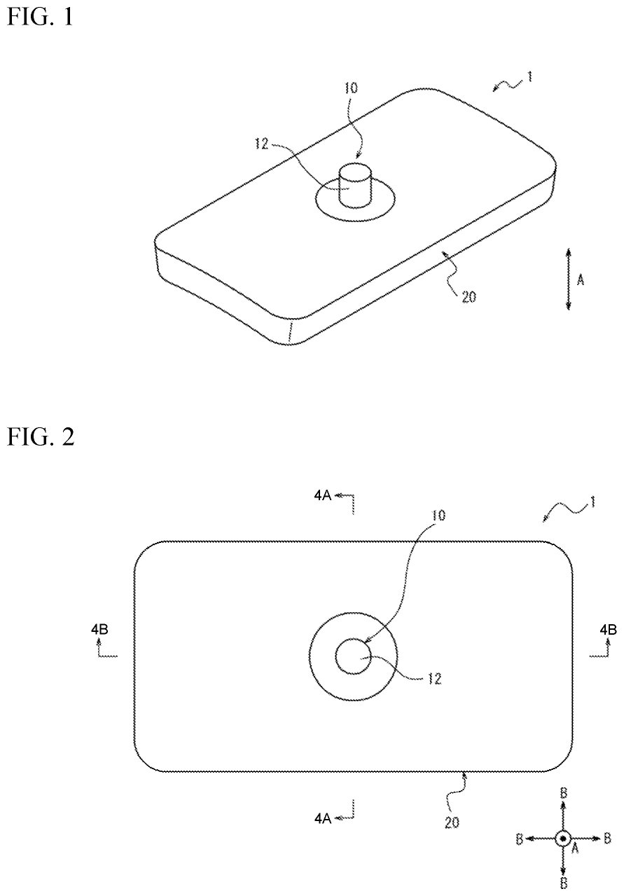

FIG. 1 is a perspective view illustrating a dry electrode 1 as one embodiment of a dry electrode according to this disclosure. FIG. 2 is a top view of the dry electrode 1. As illustrated in FIGS. 1 and 2, the dry electrode 1 is provided with an electrode core 10, and an electrode pad 20 that is electrically conductive with the electrode core 10.

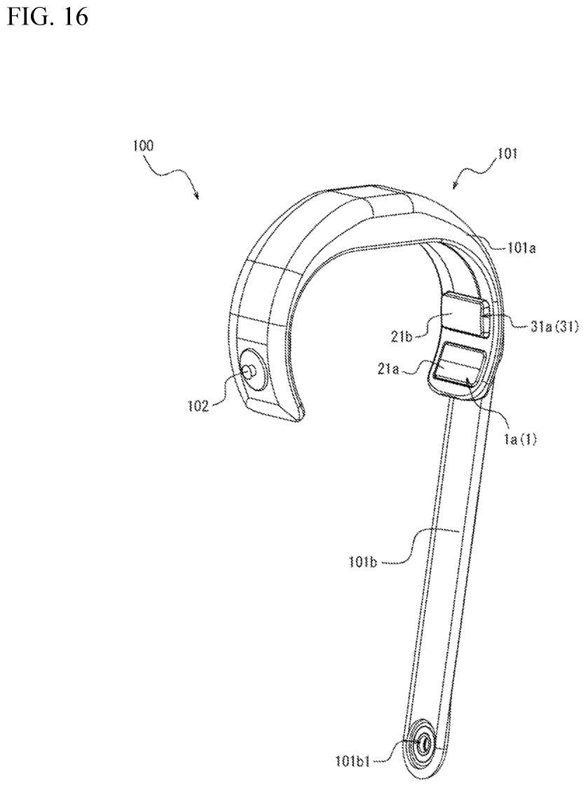

As one example, the dry electrode 1 is applied and used in a wearable device 100 (see FIGS. 16 to 18), which is described later. The wearable device 100 is mounted to and used in a predetermined position on a surface of a living body. The dry electrode 1 is configured to come into contact with the predetermined position on the surface of a living body, in a state where the wearable device 100 is mounted on (worn on) the surface of a living body. The dry electrode 1 is used, for example, in the wearable device 100, as a detecting electrode that detects a bioelectric signal such as a bioelectric current.

Hereinafter, the dry electrode 1 that is applied to the wearable device 100 will be described as an example, but the dry electrode according to this disclosure is not limited to the configuration that is applied to the wearable device 100. The dry electrode according to this disclosure may be applied to a device that is not mounted on the surface of a living body. Moreover, in the embodiment, the dry electrode 1 serving as a detecting electrode of the wearable device 100 will be described as an example, however, a usage purpose of the dry electrode according to this disclosure is not specially limited. The dry electrode 1 may be used as a stimulating electrode that applies a predetermined voltage or current to a living body, for example, in the wearable device 100.

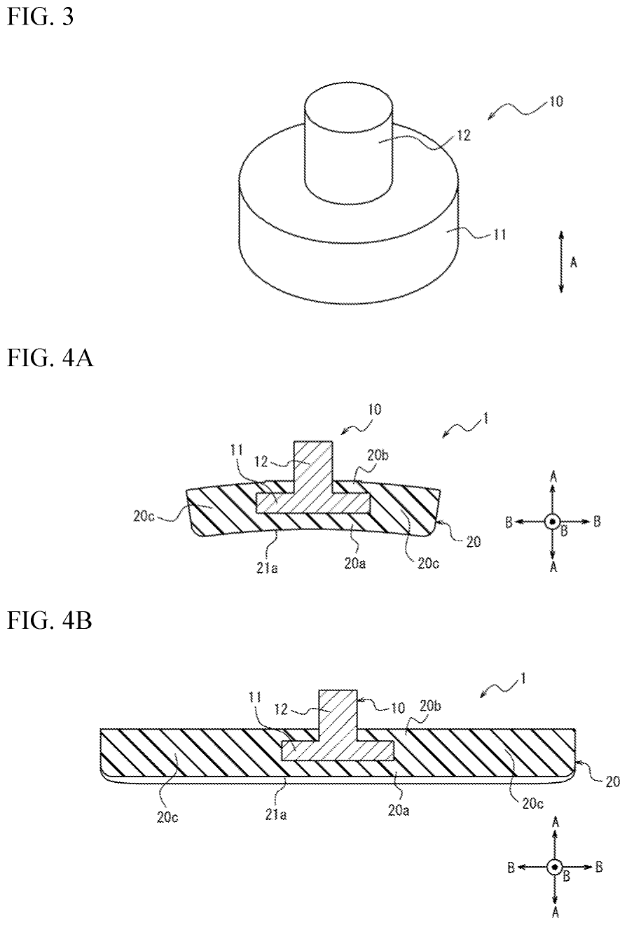

FIG. 3 is a perspective view of a simple piece of the electrode core 10. FIG. 4A is a cross-sectional view of the dry electrode 1 taken along the section line 4A-4A in FIG. 2. FIG. 4B is a cross-sectional view of the dry electrode 1 taken along the section line 4B-4B in FIG. 2.

As illustrated in FIG. 3, the electrode core 10 is provided with a plate portion 11 and a core main body portion 12. As illustrated in FIGS. 4A and 4B, the plate portion 11 is surrounded by the electrode pad 20. More specifically, the plate portion 11 in this embodiment is embedded in the electrode pad 20. The core main body portion 12 protrudes from the plate portion 11, and is at least partially exposed to an outside of the electrode pad 20.

The electrode pad 20 includes conductive rubber or a conductive elastomer having a volume resistivity of 0.5 to 10.0 Ω·cm. That is, the material forming the electrode pad 20 includes or is a conductive rubber or a conductive elastomer having a volume resistivity (electrical resistivity) of 0.5 to 10.0 Ω·cm. The dry electrode 1 is used by bringing the electrode pad 20 into contact with the surface of a living body. The use of such the electrode pad 20 can implement the dry electrode 1 capable of improving the adhesiveness with the skin.

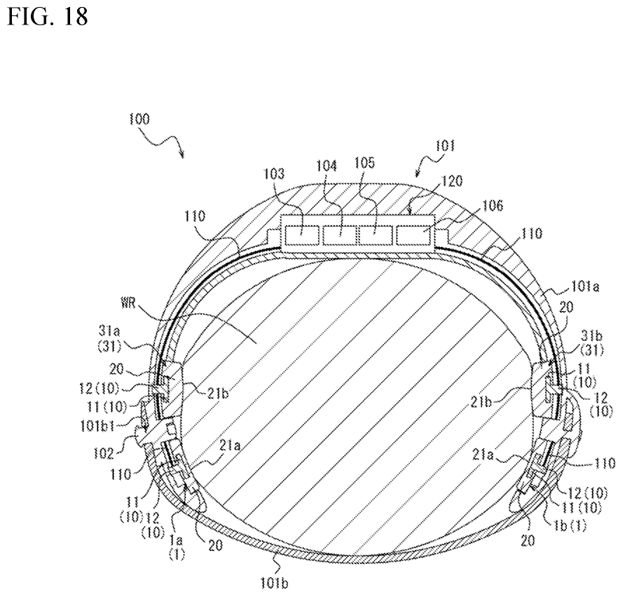

Although the details are described later, in the wearable device 100 (see FIGS. 16 to 18) to which the dry electrode 1 is applied, a portion that is exposed to the outside of the electrode pad 20, of the core main body portion 12, is electrically connected to a control unit 103 (see FIG. 18) via an electric signal line 110 (see FIG. 18). Accordingly, a bioelectric signal is transmitted from the electrode pad 20 that comes into contact with the surface of a living body through the electrode core 10 to the control unit 103.

Hereinafter, with reference to FIGS. 1 to 4B, details of the dry electrode 1 in the embodiment will be described.

### Electrode Core 10

As described above, the electrode core 10 is provided with the plate portion 11 and the core main body portion 12. The plate portion 11 in the embodiment has an outer shape that is a circular shape in a plan view (see FIG. 2) seen from a thickness direction A (i.e., as seen from a position looking at the free end of the core main body portion 12 as in FIG. 2). In this manner, the plate portion 11 is made to have the outer shape with no corner in a plan view, so that the conductivity of the plate portion 11 can be increased. The outer shape with no corner in a plan view is not limited to the circular shape in the embodiment, and may be an oval shape.

Moreover, as illustrated in FIGS. 4A and 4B, the plate portion 11 in the embodiment is surrounded by the electrode pad 20. More specifically, the electrode pad 20 surrounds both sides of the plate portion 11 in the thickness direction A, and the whole region of the plate portion 11 in a direction B (hereinafter, described as “the in-plane direction B of the plate portion 11”) orthogonal to the thickness direction A. This can prevent the electrode core 10 from pulling out from the electrode pad 20. Such configuration can be implemented, for example, by insert molding the electrode core 10 and the electrode pad 20 such that the plate portion 11 is embedded in the electrode pad 20.

In a case of the wearable device 100 (see FIGS. 16 to 18) that is configured to be mounted on a wrist, which is described later, the surface area of surfaces positioned on the both sides of the plate portion 11 in the thickness direction A is preferably 10 to 50 mm², and especially preferably set to 15 to 25 mm². This can increase the detection accuracy of an electric signal of a living body through the electrode pad 20 while downsizing (reducing the size of) the wearable device 100.

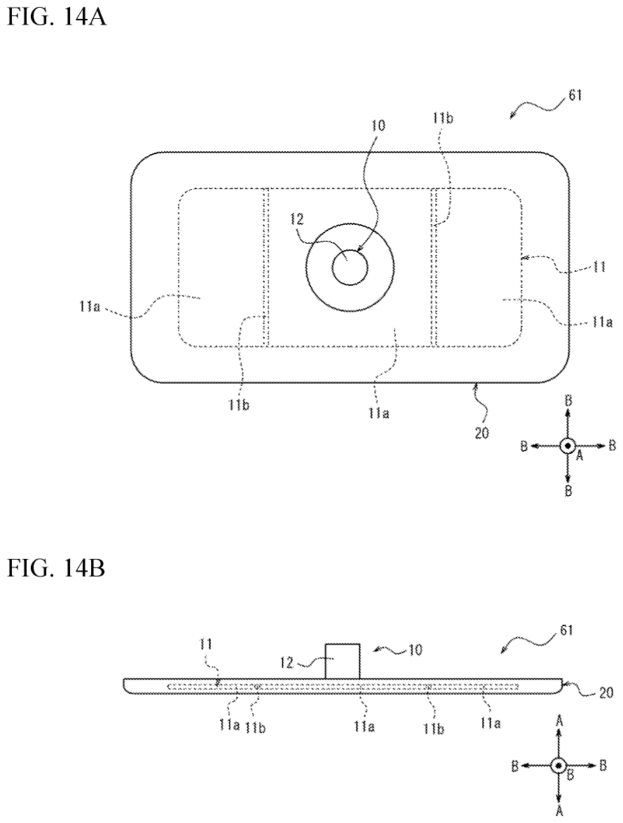

Moreover, the plate portion 11 is preferably configured to be bending deformable (bendable) in the thickness direction A. With this configuration, the plate portion 11 can be deformed and can follow the electrode pad 20 that comes into contact with a surface of a living body. Accordingly, it is possible to improve the followability of the electrode pad 20 to the surface of a living body. That is, the electrode pad 20 is better able to conform to the surface of a living body. The plate portion 11 that is bending deformable may be configured to have a low flexural rigidity as a whole and be easily bending deformed, for example. Moreover, the plate portion 11 that is bending deformable may be a plate portion including a bending portion such as a hinge, for example. A specific example thereof will be described later (see FIGS. 14A and 14B).

Recessed and protruding portions may be provided on a surface of the plate portion 11 of the electrode core 10. Providing the recessed and protruding portions on the surface of the plate portion 11 can have a larger surface area, as compared with the configuration having no recessed and protruding portions. This can attain a large contact area with the electrode pad 20. Accordingly, it is possible to attain the large friction resistance and the like between the plate portion 11 of the electrode core 10 and the electrode pad 20, and prevent the electrode core 10 from falling off from the electrode pad 20. The recessed and protruding portions can include, for example, granular or ribbed protrusions, dimpled or grooved recesses, and the like. Moreover, the plate portion 11 may be sectioned to have a through hole into which the electrode pad 20 enters. In addition, the plate portion 11 may have a mesh shape or a scaffold shape in which a plurality of through holes are sectioned. In this manner, the electrode pad 20 enters into the through hole of the plate portion 11, and the plate portion 11 and the electrode pad 20 mesh with each other, so that a binding force between the plate portion 11 and the electrode pad 20 can be increased.

The core main body portion 12 in the embodiment protrudes from the plate portion 11. More specifically, the core main body portion 12 in the embodiment protrudes from the plate portion 11 toward one side in the thickness direction A. Moreover, the core main body portion 12 in the embodiment protrudes outward further than the electrode pad 20 in the surrounding of the plate portion 11. Accordingly, the core main body portion 12 is at least partially exposed to the outside of the electrode pad 20. In the present specification, hereinafter, for convenience of explanation, a side in the thickness direction A of the plate portion 11 at which the core main body portion 12 protrudes is called “proximal side” or “upper side” in some cases. Moreover, a side in the thickness direction A of the plate portion 11 opposed to the side at which the core main body portion 12 protrudes is called “distal side” or “lower side” in some cases. In addition, a plan view in which the dry electrode 1 is seen from the thickness direction A of the plate portion 11 is simply described as “plan view of the dry electrode 1”. FIG. 2 illustrates a top surface view in which the dry electrode 1 is seen from the proximal side (upper side) toward the distal side (lower side) in the thickness direction A of the plate portion 11, in the plan view of the dry electrode 1.

The core main body portion 12 in the embodiment has a circular outer shape in a plan view of the dry electrode 1 (see FIG. 2). More specifically, the core main body portion 12 in the embodiment has a substantially cylindrical outer shape, a central axis direction of which is the thickness direction A of the plate portion 11. In other words, the plate portion 11 is concentrically continuous with one end in the central axis direction of the core main body portion 12.

A portion of the core main body portion 12 that is exposed to the outside of the electrode pad 20 is electrically connected to the control unit 103 (see FIG. 18) of the wearable device 100 (see FIGS. 16 to 18). The core main body portion 12 in the embodiment may be provided with an electric connection portion to which an electric signal line can be connected. In other words, the core main body portion 12 in the embodiment may be electrically connected to the control unit 103 via the electric signal line that is connected to the electric connection portion by soldering, for example.

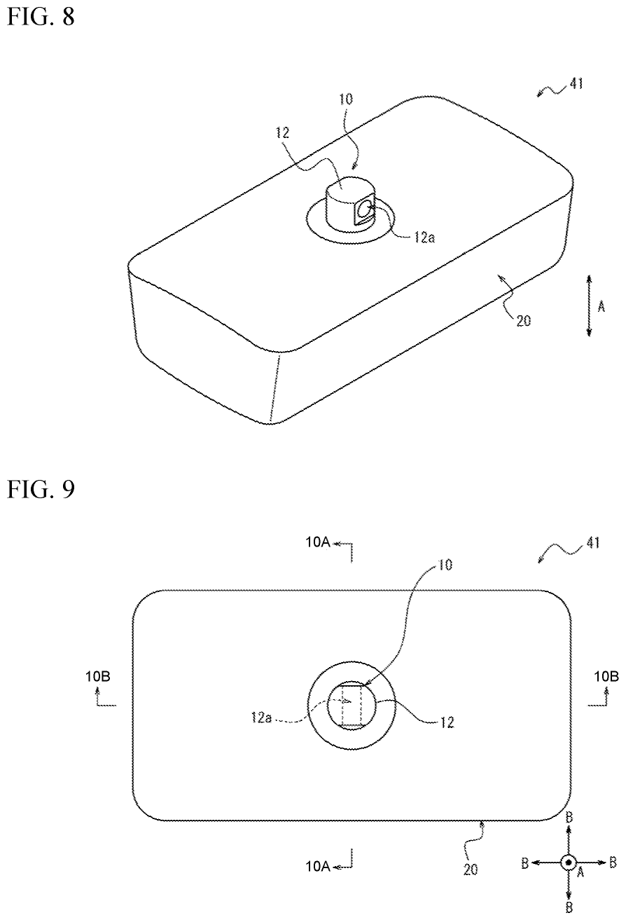

The core main body portion 12 does not need to include a specific electric connection portion. In other words, the electric signal line may be connected by soldering and the like to a portion of the core main body portion 12, that is exposed to the outside of the electrode pad 20. The core main body portion 12 is preferably provided with the electric connection portion. The core main body portion 12 is provided with the electric connection portion in accordance with a connection member such as an electric signal line to allow easy connection work of the connection member. Moreover, a physical and electrical connection state of the core main body portion 12 and the connection member such as the electric signal line is easily stabilized. A specific example of the electric connection portion of the core main body portion 12 is described later (see FIGS. 8 to 13B).

The electrode core 10 is a conductive member including metal or other conductive materials. The electrode core 10 includes, for example, gold, a gold alloy, silver, a silver alloy, copper, a copper alloy, aluminum, an aluminum alloy, zinc, nickel, brass, bronze stainless steel, and carbon. In particular, from the viewpoint of the conductivity, the electrode core 10 preferably includes at least one among gold, a gold alloy, silver, a silver alloy, copper, a copper alloy, stainless steel, and carbon. In addition, from the viewpoint of the conductivity and the chemical stability, the electrode core 10 preferably includes gold at least on an outer surface thereof. The electrode core 10 can have a configuration in which brass is coated with nickel, and a surface thereof is further coated with gold, for example. In other words, the electrode core 10 can have a configuration in which a nickel intermediate layer is laminated on an outer surface of a brass core, and a gold surface layer is further laminated on an outer surface of the nickel intermediate layer.

### Electrode Pad 20

The electrode pad 20 can be a cast of conductive rubber or a thermoplastic conductive elastomer, for example. Examples of the conductive rubber can include conductive rubber made of silicone. Moreover, examples of the conductive elastomer can include a styrene conductive elastomer. The abovementioned volume resistivity of 0.5 to 10.0 Ω·cm of the electrode pad 20 can be implemented by mixing a conductive carbon material into the abovementioned rubber material or elastomer material, for example. In the case of the wearable device 100 (see FIGS. 16 to 18) that is mounted on the wrist, which is described later, the volume resistivity of the electrode pad 20 is more preferably 0.5 to 5.0 Ω·cm, and is especially preferably 0.5 to 3.0 Ω·cm.

Moreover, the Shore A hardness of the electrode pad 20 is preferably 50 to 80. The electrode pad 20 exhibits the abovementioned hardness, whereby the followability of the electrode pad 20 to the surface of the living body can be increased. The compatibility of the range of the abovementioned volume resistivity of the electrode pad 20 and the range of the abovementioned Shore A hardness of the electrode pad 20 can be implemented, for example, by adjusting the mixing amount of the conductive carbon material relative to the rubber material such as silicone or the elastomer material such as the styrene elastomer material.

The outer surface of the electrode pad 20 is provided with a contact surface 21a that comes into contact with the surface of a living body. The contact surface 21a of the electrode pad 20 in the embodiment that comes into contact with the surface of a living body is a surface that is positioned at a distal side in the thickness direction A relative to the plate portion 11 of the electrode core 10. The surface area of the contact surface 21a of the electrode pad 20 is larger than the surface area of the surface at the distal side in the thickness direction A of the plate portion 11. In the case of the wearable device 100 (see FIGS. 16 to 18) that is mounted on the wrist, which is described later, the surface area of the contact surface 21a of the electrode pad 20 is preferably 150 to 300 mm². The resistivity of the electrode pad 20 may be higher than the resistivity of the electrode core 10. An impedance of an interface between the skin and the electrode may be ignored in a high frequency range. Therefore, the impedance of the electrode pad 20 may be selected so as to have a small influence thereof all over the high frequency range. Recessed and protruding portions may be provided on the contact surface 21a of the electrode pad 20. The recessed and protruding portions may be provided, for example, for enlarging a contact area of the contact surface 21a of the electrode pad 20 with the surface of the living body, or for improving the comfortableness in a subject when the contact surface 21a comes into contact with the subject. The recessed and protruding portions can include, for example, granular or ribbed protrusions, dimpled or grooved recesses, and the like.

The electrode pad 20 may be insert molded so as to cover a distal side of the electrode core 10, as described above. The electrode core 10 may be press-fitted into the electrode pad 20, thereby being fixed to the electrode pad 20.

More specifically, the electrode pad 20 in the embodiment is provided with a distal coating portion 20a that is positioned at the distal side in the thickness direction A relative to the plate portion 11 of the electrode core 10. The distal coating portion 20a in the embodiment covers the whole region of the surface of the plate portion 11 at the distal side. In the embodiment, the abovementioned contact surface 21a of the electrode pad 20 includes the surface of the distal coating portion 20a at the distal side. In other words, the contact surface 21a of the electrode pad 20 in the embodiment includes a lower surface of the distal coating portion 20a that is positioned at an opposite side of a top surface thereof that is opposed to the plate portion 11.

As illustrated in FIG. 4A, the contact surface 21a of the electrode pad 20 in the embodiment includes a recessed curved surface. More specifically, the recessed contact surface 21a of the electrode pad 20 in the embodiment includes a circular arc-shaped groove having a cross-sectional shape of a circular arc shape. In other words, the contact surface 21a in the embodiment forms a circular arc shape (concave shape) in a cross-sectional view along an up-and-down direction on a plane of paper in FIG. 2, including the cross-sectional view illustrated in FIG. 4A. In contrast, the contact surface 21a in the embodiment forms a straight line shape in a cross-sectional view along a left-and-right direction on a plane of paper in FIG. 2, including the cross-sectional view illustrated in FIG. 4B. The electrode pad 20 in the embodiment is disposed such that the circumferential direction of the circular arc-shaped groove as the contact surface 21a is along the circumferential direction of the wrist, in a state where the wearable device 100 (see FIGS. 16 to 18) is mounted on the wrist, which is described later. That is, the circumferential direction of the circular arc-shaped contact surface 21a is oriented along the circumferential direction of the wrist. Accordingly, it is possible to dispose the contact surface 21a of the electrode pad 20 along the wrist surface as a surface of a living body, and secure a large contact area with the contact surface 21a.

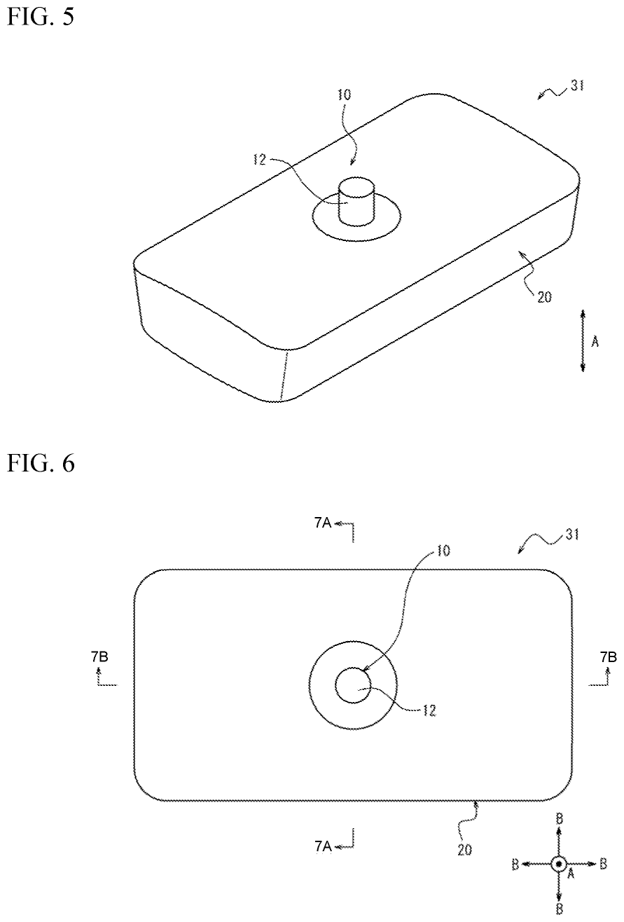

In a case where the electrode pad 20 includes conductive rubber or a conductive elastomer having a volume resistivity of 0.5 to 10.0 Ω·cm, a contact surface of the electrode pad 20 preferably includes a protruding surface. The conductive rubber or the conductive elastomer having a volume resistivity of 0.5 to 10.0 Ω·cm can secure the flexibility to follow a surface of a living body by having Shore A hardness of 50 to 80, for example, however, the sufficient flexibility to follow the surface of a living body cannot be secured if the conductive rubber or the conductive elastomer is harder than the Shore A hardness of 50 to 80 in some cases. In that case, the recessed curved surface as the contact surface 21a does not follow the surface of a living body at all in accordance with a physical size and the like of a subject, and a necessary contact area cannot be obtained on the contrary in some cases. Accordingly, independent of the hardness of the electrode pad 20 and the physical size and the like of the subject, from the viewpoint of securing a desired contact area, the contact surface of the electrode pad 20 preferably includes the protruding surface. A specific example in which the contact surface includes the protruding surface is described later (see FIGS. 5 to 13B).

Moreover, the electrode pad 20 in the embodiment is provided with a proximal coating portion 20b that is positioned at a proximal side in the thickness direction A relative to the plate portion 11 of the electrode core 10. The proximal coating portion 20b extends in the in-plane direction B so as to surround the circumference of the core main body portion 12. Moreover, the proximal coating portion 20b covers the whole region of the surface of the plate portion 11 at the proximal side in the surrounding of the core main body portion 12. In other words, the core main body portion 12 in the embodiment protrudes to the proximal side further than the proximal coating portion 20b from the plate portion 11 so as to penetrate through the proximal coating portion 20b of the electrode pad 20.

In addition, the electrode pad 20 in the embodiment is provided with a side coating portion 20c that covers the whole surrounding region of the plate portion 11 of the electrode core 10 in the in-plane direction B. In other words, the side coating portion 20c covers the whole surrounding region of the plate portion 11 in a plan view of the dry electrode 1 (see FIG. 2). Moreover, the side coating portion 20c is continuous with the distal coating portion 20a at the distal side in the whole surrounding region of the plate portion 11 in the in-plane direction B. In addition, the side coating portion 20c is continuous with the proximal coating portion 20b at the proximal side in the whole surrounding region of the plate portion 11 in the in-plane direction B.

In this manner, the electrode pad 20 in the embodiment covers the whole region of the plate portion 11 of the electrode core 10, except the position where the core main body portion 12 is provided. Thus, as shown in FIGS. 4A and 4B, the entire outer surface of the plate portion 11 of the electrode core 10 and a portion of the outer surface of the core main body portion 12 located most adjacent to the plate portion 11 is covered or surrounded by the electrode pad 20.

### Second Embodiment

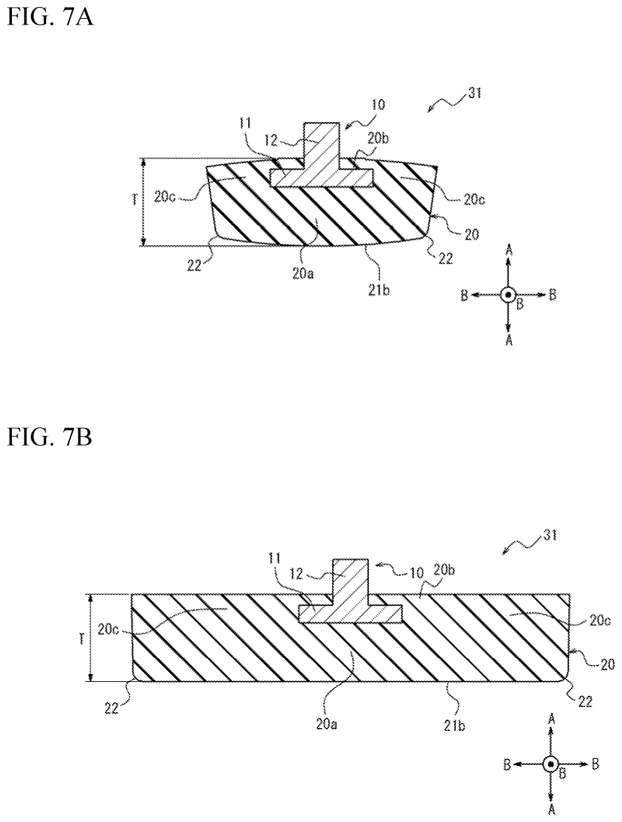

Next, with reference to FIGS. 5 to 7B, a dry electrode 31 as a second embodiment will be described. FIG. 5 is a perspective view of the dry electrode 31. FIG. 6 is a top view of the dry electrode 31. FIG. 7A is a cross-sectional view of the dry electrode 31 taken along the section line 7A-7A in FIG. 6. FIG. 7B is a cross-sectional view of the dry electrode 31 taken along the section line 7B-7B in FIG. 6.

When the dry electrode 31 in the embodiment is compared with the abovementioned dry electrode 1 (see FIG. 1 and the like), the configuration of the contact surface of the electrode pad 20 is mainly different from each other. Herein, only the difference between the dry electrode 31 and the previously-described dry electrode 1 will be described, and a detailed description of aspects of the dry electrode that are common to both embodiments will not be repeated.

The contact surface 21a of the electrode pad 20 (see FIG. 4A) in the abovementioned dry electrode 1 includes a circular arc-shaped groove (concave groove). In contrast, a contact surface 21b of the electrode pad 20 in the dry electrode 31 in this embodiment includes a protruding surface or convex surface. In other words, a surface of the electrode pad 20 at the distal side in the dry electrode 31 in the embodiment includes the protruding surface.

Specifically, as illustrated in FIG. 7A, the contact surface 21b of the electrode pad 20 in the embodiment includes a protruding curved surface. More specifically, the contact surface 21b of the electrode pad 20 in the embodiment includes a circular arc-shaped protruding surface that extends in a ridge shape, and has a cross-sectional shape of a circular arc shape along a width direction orthogonal to an extending direction. In other words, the contact surface 21b in the embodiment forms a circular arc shape in a cross-sectional view along the up-and-down direction on a plane of paper in FIG. 6, including the cross-sectional view illustrated in FIG. 7A. In contrast, the contact surface 21b in the embodiment forms a straight line shape in a cross-sectional view along the left-and-right direction on a plane of paper in FIG. 6 (hereinafter, simply described as “cross-sectional view in the width direction of the contact surface 21b”), including the cross-sectional view illustrated in FIG. 7B. The electrode pad 20 in the embodiment is disposed such that the circumferential direction of the circular arc-shaped protruding surface as the contact surface 21b is along the circumferential direction of the wrist, in a state where the wearable device 100 (see FIGS. 16 to 18) is mounted on the wrist, which is described later. Accordingly, independent of the hardness of the electrode pad 20 and the physical size and the like of the subject, it is possible to reliably bring at least a top portion of the contact surface 21b of the electrode pad 20 into contact with the wrist surface as the surface of a living body. Accordingly, a desired contact area can be reliably secured.

The dry electrode 31 in the embodiment is applied to the wearable device 100 (see FIGS. 16 to 18) that is mounted on a wrist, which is described later. Accordingly, the protruding surface as the contact surface 21b of the electrode pad 20 in the embodiment is made to have a circular arc-shaped protruding surface that extends in a ridge shape. The shape of the protruding surface as the contact surface 21b can be changed as appropriate in accordance with the configuration of the wearable device, a mounting position of the wearable device on the surface of a living body, and the like. The protruding surface as the contact surface 21b may include, for example, a polygonal protruding surface that extends in a ridge shape, and has a cross-sectional shape of a polygonal shape along the width direction orthogonal to the extending direction. Moreover, the protruding surface as the contact surface 21b may have a cone-shaped or frustum-shaped outer shape in which the surface is raised from the outer edge portion in the surrounding in a plan view of the dry electrode 31 (see FIG. 6) toward the central portion, and the top portion includes a protruding curved surface or plane, for example. In any of the abovementioned shapes, the top portion of the contact surface 21b that comes into contact with the surface of a living body preferably includes a protruding curved surface. Accordingly, unpleasant feeling of the subject can be reduced.

Moreover, an outer surface of the distal coating portion 20a as the contact surface 21b in the embodiment and an outer surface of the side coating portion 20c are smoothly continuous with each other with a protruding curved surface 22. Accordingly, even in a case where the whole region of the outer surface of the distal coating portion 20a comes into contact with the surface of a living body, with the corner portion between the outer surface of the distal coating portion 20a and the outer surface of the side coating portion 20c, it is possible to reduce the possibility of pain and unpleasant feeling to the subject. The curvature radius of the protruding curved surface 22 between the outer surface of the distal coating portion 20a and the outer surface of the side coating portion 20c is smaller than the curvature radius at a position including the top portion (most protruding portion) of the circular arc-shaped protruding surface as the contact surface 21b. In the case of the wearable device 100 (see FIGS. 16 to 18) that is mounted on the wrist, which is described later, the curvature radius of the protruding curved surface 22 is preferably 0.5 to 4.0 mm, and is more preferably 1.0 to 2.0 mm.

In the embodiment, a maximum thickness T of the electrode pad 20 in the thickness direction A may be 2 to 6 mm. The maximum thickness T is not specially limited. The maximum thickness T can be changed as appropriate in accordance with the configuration of the wearable device, the mounting position of the wearable device on the surface of a living body, and the like.

### Third Embodiment

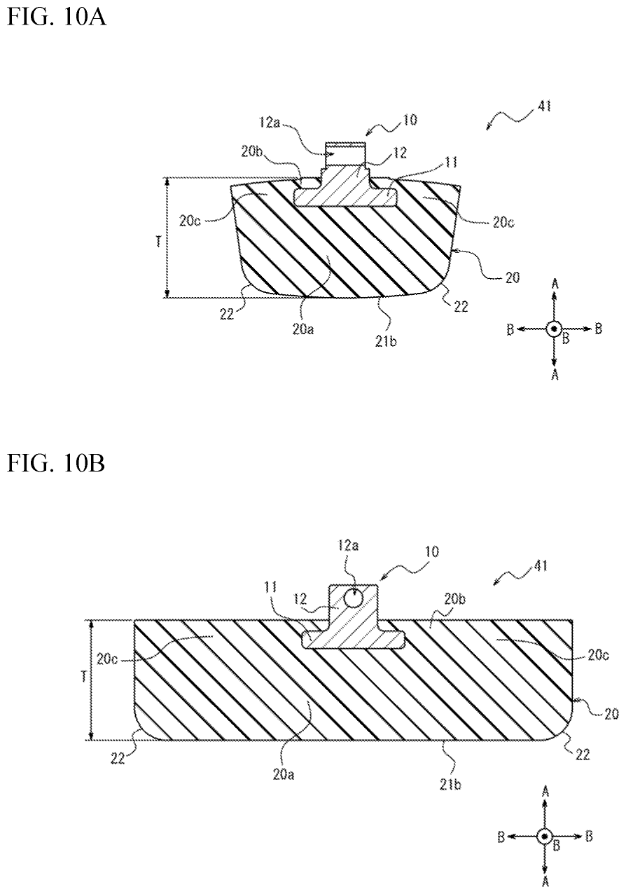

Next, with reference to FIGS. 8 to 10B, a dry electrode 41 as a third embodiment will be described. FIG. 8 is a perspective view of the dry electrode 41. FIG. 9 is a top view of the dry electrode 41. FIG. 10A is a cross-sectional view of the dry electrode 41 taken along the section line 10A-10A in FIG. 9. FIG. 10B is a cross-sectional view of the dry electrode 41 taken along the section line 10B-10B in FIG. 9.

When the dry electrode 41 in the embodiment is compared with the abovementioned dry electrode 31 (see FIG. 5 and the like), the configuration of the core main body portion 12 of the electrode core 10 is mainly different from each other. Herein, only the difference between the dry electrode 41 and the previously-described dry electrodes will be described, and a detailed description of aspects of the dry electrode that are e common to the embodiments will not be repeated.

The core main body portion 12 of the electrode core 10 in the embodiment is provided with an electric connection portion to which a connection member such as an electric signal line can be connected. More specifically, in the core main body portion 12 of the electrode core 10 in the embodiment, a through hole 12a is provided at a position where the core main body portion 12 is exposed from the electrode pad 20. A connection member such as an electric signal line is fixed to the core main body portion 12 by soldering and the like, in a state where the connection member is inserted into or positioned in the through hole 12a in the core main body portion 12, for example. The through hole 12a is provided to allow easy connection work of a connection member such as an electric signal line. Moreover, a physical and electrical connection state of the core main body portion 12 and the connection member such as the electric signal line becomes easily stabilized.

The through hole 12a in the embodiment is a circular cross-sectional hole that penetrates through the substantially cylindrical core main body portion 12 in a direction orthogonal to the central axis direction of the core main body portion 12. Moreover, the diameter of the through hole 12a in the embodiment may be 0.5 to 1.0 mm. The arrangement position, the cross-sectional shape, and the diameter of the through hole 12a are not specially limited.

### Fourth Embodiment

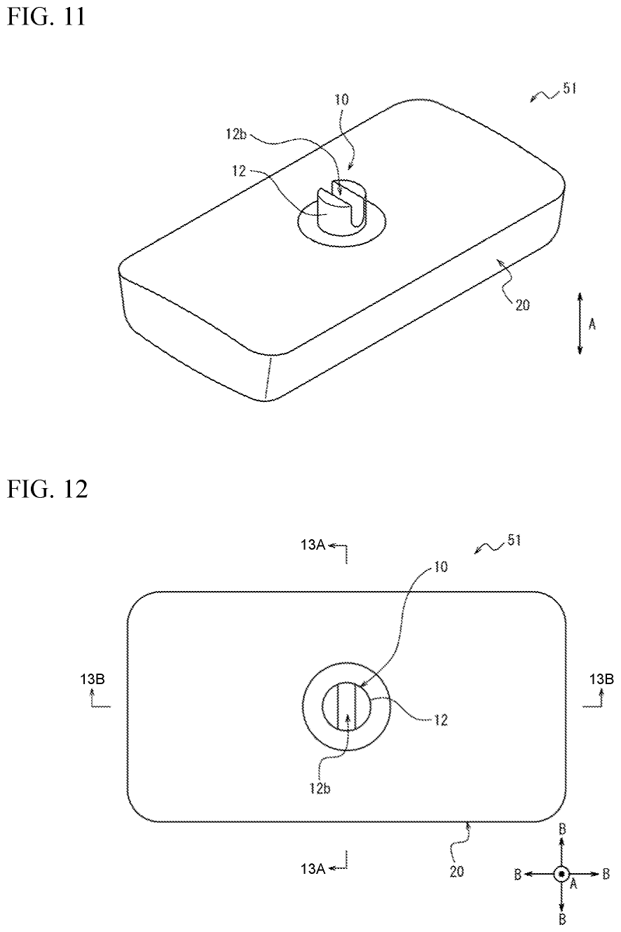

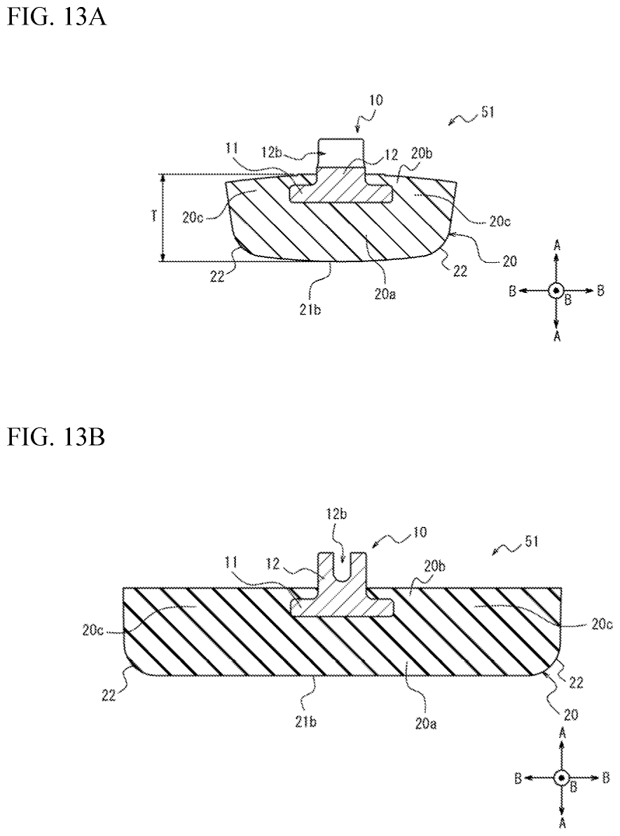

Next, with reference to FIGS. 11 to 13B, a dry electrode 51 as a fourth embodiment will be described. FIG. 11 is a perspective view of the dry electrode 51. FIG. 12 is a top view of the dry electrode 51. FIG. 13A is a cross-sectional view of the dry electrode 51 along the section line 13A-13A in FIG. 12. FIG. 13B is a cross-sectional view of the dry electrode 51 along the section line 13B-13B in FIG. 12.

When the dry electrode 51 in the embodiment is compared with the abovementioned dry electrode 41 (see FIG. 8 and the like), the configuration of the core main body portion 12 of the electrode core 10 is different from each other. Herein, only the difference between the dry electrode 51 and the previously-described dry electrode 41 will be described, and a detailed description of aspects of the dry electrode that are common to the embodiments will not be repeated.

The core main body portion 12 of the electrode core 10 in the embodiment is provided with an electric connection portion to which a connection member such as an electric signal line can be connected. More specifically, the outer surface of the core main body portion 12 of the electrode core 10 in the embodiment is provided with a recessed portion 12b at a position where the core main body portion 12 is exposed from the electrode pad 20. The recessed portion 12b in the embodiment is a groove that is formed in a proximal end face of the core main body portion 12. The groove as the recessed portion 12b is a through groove that is continuously connected with or to an outer edge on the proximal end face of the substantially cylindrical core main body portion 12. Moreover, the groove as the recessed portion 12b extends in a substantially linear shape. In addition, the groove as the recessed portion 12b has an outer shape having a substantially rectangular cross-sectional shape. A connection member such as an electric signal line is fixed to the core main body portion 12 by soldering and the like, in a state where the connection member is disposed in the recessed portion 12b in the core main body portion 12, for example. Such the recessed portion 12b is provided to allow easy connection work of a connection member such as an electric signal line. Moreover, a physical and electrical connection state of the core main body portion 12 and the connection member such as the electric signal line becomes easily stabilized.

The recessed portion 12b in the embodiment is a through groove having a rectangular cross-sectional shape that extends linearly in the proximal end face of the substantially cylindrical core main body portion 12, and the arrangement position, the extending direction, and the cross-sectional shape thereof are not specially limited. Moreover, the groove width of the groove as the recessed portion 12b in the embodiment may be 0.5 to 1.0 mm, however, the groove width is not specially limited either.

### Fifth Embodiment

Next, with reference to FIGS. 14A and 14B, a dry electrode 61 as a fifth embodiment will be described. FIG. 14A is a top view of the dry electrode 61. FIG. 14B is a side view of the dry electrode 61. In FIGS. 14A and 14B, for convenience of explanation, a position of the plate portion 11 of the electrode core 10 is illustrated by a dashed line.

When the dry electrode 61 in the embodiment is compared with the abovementioned dry electrode 1 (see FIG. 1 and the like), the configuration of the plate portion 11 of the electrode core 10 is the main difference between the two embodiments. Herein, only the difference between the dry electrode 61 and the previously-described dry electrode 1 will be described, and a detailed description of aspects of the dry electrode that are common to the embodiments will not be set forth.

The plate portion 11 in the embodiment is configured to be bending deformable (bendable) in the thickness direction A. With this configuration, the plate portion 11 can be deformed by following the electrode pad 20 that comes into contact with a surface of a living body. Accordingly, it is possible to improve the followability of the electrode pad 20 to the surface of a living body. That is, the electrode pad 20 is better able to conform to the surface of a living body. More specifically, the plate portion 11 in the embodiment is provided with a plurality of rectangular plate-like (plate-shaped) flat plate portions 11a, and bending portions 11b each of which is positioned between two adjacent flat plate portions 11a. In other words, the adjacent two flat plate portions 11a are continuous with each other with the bending portion 11b therebetween. The bending portion 11b has a flexural rigidity smaller than that at any position of the flat plate portion 11a. Accordingly, the plate portion 11 is configured to be bending deformable (bendable) at the position of the bending portion 11b with higher priority than the arbitrary position of the flat plate portion 11a. The configuration of the bending portion 11b is not specially limited, but the bending portion 11 may include a hinge, for example. The bending portion 11b is formed integrally with the flat plate portion 11a, for example, and may include a thin-walled portion having a thickness in the thickness direction A thinner than that at another position of the flat plate portion 11a. In this manner, the plate portion 11 easily bending deforms such that the position of the bending portion 11b serves as a polygonal line. Moreover, the bending portion 11b may include a member that is different from the flat plate portion 11a, and connects the adjacent two flat plate portions 11a to each other.

In the embodiment, three flat plate portions 11a are arranged in series. Moreover, each of the bending portions 11b is provided between each of the adjacent two flat plate portions 11a among the three flat plate portions 11a arranged in series. The number and the arrangement position of each of the flat plate portions 11a and the bending portions 11b can be changed as appropriate in accordance with the configuration of the wearable device, the mounting position of the wearable device on the surface of a living body, and the like.

Moreover, the core main body portion 12 protrudes from the flat plate portion 11a that is positioned at the center among the three flat plate portions 11a arranged in series, however, the configuration of the arrangement of the core main body portion 12 is not limited to this configuration.

### Sixth Embodiment

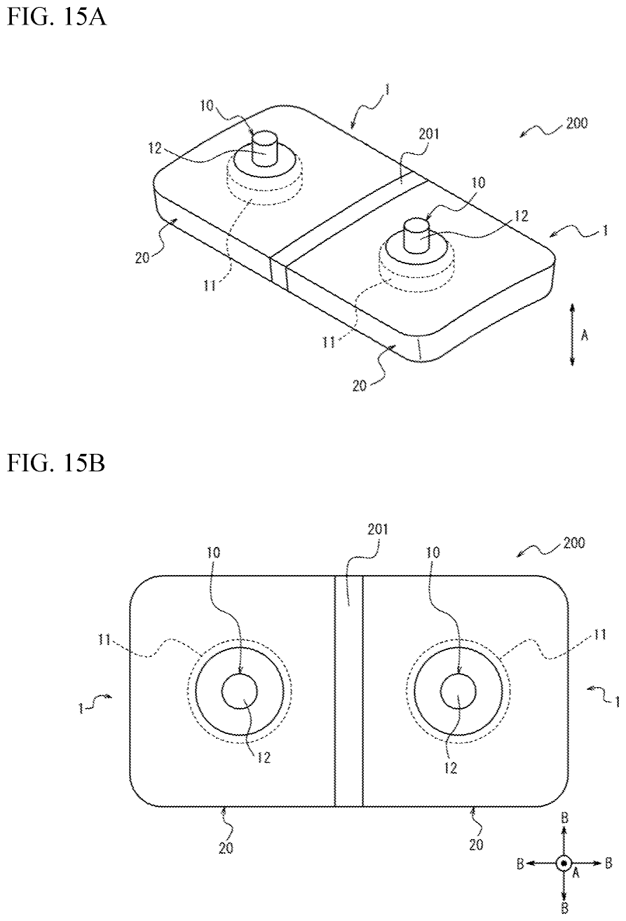

Next, with reference to FIGS. 15A and 15B, a dry electrode array 200 in which a plurality of the dry electrodes 1 are interlocked will be described. FIG. 15A is a perspective view of the dry electrode array 200. FIG. 15B is a top view the dry electrode array 200. Herein, the dry electrode array 200 in which two dry electrodes 1 (see FIG. 1 and the like) in the first embodiment are interlocked will be described as an example. However, the dry electrode array in which a plurality of any of the electrodes 31, 41, 51, and 61, which are respectively illustrated in the second to fifth embodiments, are interlocked may be used.

In the dry electrode array 200 illustrated in FIGS. 15A and 15B, the two dry electrodes 1 are interlocked with an insulation portion 201 therebetween. More specifically, the insulation portion 201 of the dry electrode array 200 interlocks the electrode pads 20 of the two dry electrodes 1 to each other. The two dry electrodes 1 of the dry electrode array 200 are interlocked with only the insulation portion 201 therebetween, and do not conduct with each other (i.e., are not electrically conductive with each other). Of course, the two dry electrodes 1 may be conducted. That is, the two dry electrodes 1 may be electrically conductive with each other. The configuration and the material of the insulation portion 201 are not specially limited. Moreover, in the dry electrode array 200 illustrated in FIGS. 15A and 15B, the two dry electrodes 1 are interlocked, however, three or more dry electrodes 1 may be interlocked, and the number of dry electrodes is not specially limited.

### Seventh Embodiment

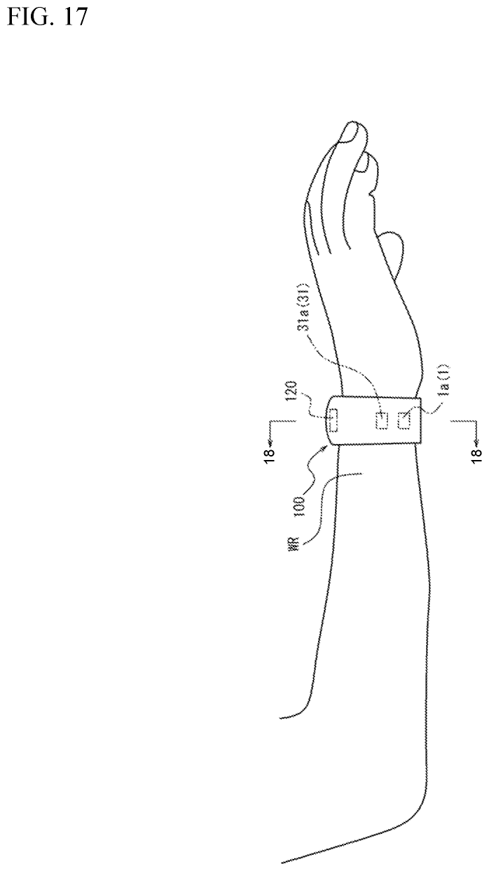

Lastly, with reference to FIGS. 16 to 18, the wearable device 100 as one embodiment of a wearable device according to this disclosure will be described. FIG. 16 is a perspective view of the wearable device 100. FIG. 17 is a use state view illustrating a state where the wearable device 100 is mounted on a wrist WR of a subject so as to cover his/her skin. FIG. 18 is a cross-sectional view along the section line 18-18 in FIG. 17.

The wearable device 100 in the embodiment can detect a bioelectric signal. The wearable device 100 is mounted on a heart failure patient, for example, for several hours almost every day or for a short time of several minutes to several tens of minutes almost every day, during a medium-to-long period of time of several months and one year or more, and thus is used for the state management or condition management (e.g., management of the state or condition of a heart failure patient) for the heart failure patient during that time. In other words, by monitoring bioelectric signals from the heart failure patient with the wearable device 100, it is possible to find a sign that the heart failure becomes worse at an early stage. The usage purpose of the wearable device 100 is not limited to the state management for the heart failure patient. The wearable device 100 may be used for another usage purpose, for example, the moisture management for a dialysis patient, or the state management of an edema after the surgery for cancer.

As illustrated in FIGS. 16 to 18, the wearable device 100 is provided with one or more dry electrodes. The dry electrode included in the wearable device 100 may be any of the dry electrodes illustrated as examples in the abovementioned first to fifth embodiments. Moreover, when the wearable device 100 is provided with a plurality of dry electrodes, all of the dry electrodes may be one kind of the dry electrodes illustrated as examples in the abovementioned first to fifth embodiments, or may be a combination of several types of the dry electrodes in the abovementioned first to fifth embodiments. In addition, the wearable device 100 may be provided with a dry electrode having a configuration different from those illustrated as examples in the abovementioned first to fifth embodiments. In addition, the plurality of the dry electrodes in the wearable device 100 may include a dry electrode array in which a plurality of dry electrodes are interlocked as indicated in the sixth embodiment.

In the embodiment, as one example, the wearable device 100 that is provided with four dry electrodes will be described as an example. Two dry electrodes among the four dry electrodes in the embodiment are the dry electrodes 1 (see FIG. 1 and the like) indicated in the first embodiment. Moreover, the remaining two dry electrodes among the four dry electrodes in the embodiment are the dry electrodes 31 (see FIG. 5 and the like) indicated in the second embodiment. In the core main body portion 12 of any of the four dry electrodes, the through hole 12a indicated in the third embodiment or the recessed portion 12b indicated in the fourth embodiment is formed. Hereinafter, for convenience of explanation, the two dry electrodes 1 in the wearable device 100 in the embodiment are described as a “first dry electrode 1a” and a “second dry electrode 1b”. Moreover, the two dry electrodes 31 in the wearable device 100 in the embodiment are described as a “third dry electrode 31a” and a “fourth dry electrode 31b”.

More specifically, the wearable device 100 in the embodiment is provided with the first to fourth dry electrodes 1a, 1b, 31a, and 31b, and a device main body 101 that holds these first to fourth dry electrodes 1a, 1b, 31a, and 31b.

The device main body 101 in the embodiment is provided with a C-character shaped housing main body 101a (C-shaped housing main body 101a) in which if formed a clearance to let the wrist WR through, and a belt body (elongated belt) 101b that is fixed to the housing main body 101a. In the illustrated embodiment shown in FIG. 16, the belt 101b is connected to and projects away from one of the ends of the C-shaped housing main body 101a or mounting portion. A proximal portion of the belt body 101b is fixed to one end portion of the housing main body 101a. A latch portion 102 capable of latching a distal end portion of the belt body 101b is provided to the other end portion of the housing main body 101a. The latch portion 102 in the embodiment includes a latch protrusion that is fitted into a through hole 101b1 formed in the distal end portion of the belt body 101b, however, the configuration of the latch portion 102 is not specially limited.

The wearable device 100 in the embodiment lets the wrist WR of the subject through the clearance of the C-shaped housing main body 101a to dispose the wrist WR of the subject in the C-shaped housing main body 101a. In that state, the belt body 101b is threaded over from the side of the one end portion of the C-shaped housing main body 101a to the side of the other end portion to latch the distal end portion of the belt body 101b to the latch portion 102 of the housing main body 101a. In this manner, the wearable device 100 in the embodiment is mounted on the wrist WR of the subject. The housing main body 101a and the belt body 101b may be configured to be capable of adjusting the width of the clearance in the housing main body 101a in accordance with the peripheral length of the wrist WR of the subject, for example.

The first to fourth dry electrodes 1a, 1b, 31a, and 31b in the embodiment are held in the housing main body 101a such that the contact surfaces 21a and 21b are exposed on an inner surface side of the C-shape of the housing main body 101a. More specifically, the first dry electrode 1a and the third dry electrode 31a in the embodiment are disposed to the one end portion of the C-shaped housing main body 101a. Moreover, the second dry electrode 1b and the fourth dry electrode 31b in the embodiment are disposed to the other end portion of the C-shaped housing main body 101a. As described above, in a state where the wearable device 100 is mounted on the circumference of the wrist WR of the subject, these first to fourth dry electrodes 1a, 1b, 31a, and 31b come into contact with the skin, as a surface of a living body, of the wrist WR. More specifically, the recessed contact surfaces 21a of the electrode pads 20 of the first dry electrode 1a and the second dry electrode 1b, and the protruding contact surfaces 21b of the electrode pads 20 of the third dry electrode 31a and the fourth dry electrode 31b come into contact with the skin of the wrist WR.

In this manner, the device main body 101 in the embodiment is provided with the C-shaped housing main body 101a as a mounting portion that can be mounted on the living body in a state where the electrode pads 20 of the first to fourth dry electrodes 1a, 1b, 31a, and 31b are brought into contact with the surface of the living body. The configuration of the mounting portion of the device main body 101 is not limited to that indicated in the embodiment. The mounting portion of the device main body 101 can be changed as appropriate in accordance with the configuration of the wearable device, the mounting position of the wearable device on the surface of a living body, and the like.

Moreover, in the wearable device 100 in the embodiment, either one group of a group of the first dry electrode 1a and the second dry electrode 1b, and a group of the third dry electrode 31a and the fourth dry electrode 31b may be used as a pair of stimulating electrodes in which a current is applied therebetween. Moreover, the other group may be used as a pair of detecting electrodes that detect bioelectric signals. Hereinafter, as one example, an example in which the pair of the first dry electrode 1a and the second dry electrode 1b is used as stimulating electrodes, and the pair of the third dry electrode 31a and the fourth dry electrode 31b is used as detecting electrodes will be described, however, the first to fourth dry electrodes 1a, 1b, 31a, and 31b may be used as dry electrodes for another usage purpose.

The device main body 101 is provided with an electronic device 120. More specifically, the electronic device 120 in the device main body 101 in the embodiment is provided with the control unit 103, a power source unit 104, a communication unit 105, and a storage unit 106. The control unit 103, the power source unit 104, the communication unit 105, and the storage unit 106 are housed in the housing main body 101a.

The control unit 103 is electrically connected to the core main body portion 12 of the electrode core 10 in each of the first to fourth dry electrodes 1a, 1b, 31a, and 31b via the electric signal line 110. The control unit 103 may control, for example, an applied current to the pair of the stimulating electrodes including the first dry electrode 1a and the second dry electrode 1b. Moreover, the control unit 103 may process bioelectric signals detected by the pair of the detecting electrodes including the third dry electrode 31a and the fourth dry electrode 31b, and measure a bioelectric impedance, for example.

The control unit 103 is one or more processors. The control unit 103 is implemented, for example, by a dedicated processor specialized in measurement of an bioelectric impedance and the like, but may be implemented by a general-purpose processor such as a central processing unit (CPU). The control unit 103 may include one or more dedicated circuits. The dedicated circuit is, for example, a field-programmable gate array (FPGA) or an application specific integrated circuit (ASIC). The control unit 103 controls the respective portions of the wearable device 100 to execute information processing relating to the motion of the wearable device 100.

The power source unit 104 provides electric power for driving the wearable device 100. The power source unit 104 may be implemented by a rechargeable battery such as a lithium ion battery, for example.

The communication unit 105 is a communication interface for communicating with an external terminal such as a PC, a tablet, or a smartphone, for example. The communication unit 105 communicates with the external terminal, and transmits a measurement value such as a bioelectric impedance to the external terminal. The communication unit 105 communicates with the external terminal using Bluetooth (registered trademark), for example, but the embodiment is not limited to this, for example, another wireless communication path such as a wireless local area network (LAN) or a wired cable may be used for the communication.

The storage unit 106 includes storage module including a random access memory (RAM) and a read-only memory (ROM), for example. The storage unit 106 may function as a main storage device or a cache memory that stores therein measurement value information such as a bioelectric impedance, for example. The storage unit 106 may store therein information that is used for the motion of the wearable device 100. The storage unit 106 may store therein, for example, various kinds of information that are received by a system program, an application program, and the communication unit 105.

The dry electrode and the wearable device according to this disclosure are not limited to the specific configuration indicated in the abovementioned embodiments, but various modifications, changes, and combinations are possible without deviating from the scope of the claims. For example, a dry electrode that has another shape and is configured by combining the respective portions in the dry electrodes 1, 31, 41, 51, and 61 indicated as the first to fifth embodiments also belongs to the technical range of this disclosure.

Moreover, the wearable device 100 indicated as the seventh embodiment is configured so as to be mountable on the wrist WR of the subject as described above, however, the wearable device according to this disclosure is not limited to the configuration. The wearable device according to this disclosure may be configured to be mounted on a position on an arm other than the wrist WR, for example. Moreover, the wearable device according to this disclosure may be configured to be mountable on a hand, a neck, a waist, a lower back, a leg including an ankle, a head, or the like. In addition, the wearable device according to this disclosure may be configured as a part of clothing such as a shirt and trousers, for example. In addition, the wearable device according to this disclosure may be configured as a part of an accessory, such as a bracelet, an anklet, an earring, a necklace, and spectacles, for example.

Moreover, in the dry electrode according to this disclosure, the additional use of a gel-type adhesive compound or the like for optimizing an impedance with the skin is not compulsory. The wearable device according to this disclosure may be provided with, in addition to the dry electrode according to this disclosure, a wet electrode that uses the gel-type adhesive compound or the like.

This disclosure relates to a dry electrode and a wearable device. The detailed description above describes embodiments of the dry electrode and the wearable device representing examples of the new dry electrode and wearable device disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A dry electrode comprising:
an electrode core;
an electrode pad that is electrically conductive with the electrode core;
the electrode core including:
a plate portion that is surrounded by the electrode pad;
a core main body portion that protrudes from the plate portion, and is at least partially exposed to an outside of the electrode pad; and
the electrode pad being comprised of conductive rubber or a conductive elastomer having a volume resistivity of 0.5 to 10.0 Ω·cm.

2. The dry electrode according to claim 1, wherein Shore A hardness of the electrode pad is 50 to 80.

3. The dry electrode according to claim 1, wherein an outer surface of the electrode pad includes a contact surface that is configured to come into contact with a surface of a living body during use of the dry electrode, and the contact surface includes a protruding portion that protrudes in a direction away form the core main body.

4. The dry electrode according to claim 1, wherein the plate portion of the electrode core has a circular outer shape in a plan view.

5. The dry electrode according to claim 1, wherein the core main body portion of the electrode core includes a through hole at a position on the core main body portion that is exposed from the electrode pad.

6. The dry electrode according to claim 1, wherein an outer surface of the core main body portion of the electrode core includes a recessed portion at a position on the core main body portion that is exposed from the electrode pad.

7. The dry electrode according to claim 1, wherein the electrode core is comprised of a material that includes at least one of gold, a gold alloy, silver, a silver alloy, copper, a copper alloy, stainless steel, and carbon.

8. The dry electrode according to claim 7, wherein at least the outer surface of the electrode core includes gold.

9. The dry electrode according to claim 1, wherein the plate portion includes at least two plate-shaped portions connected to each other by a bending portion that has a flexural rigidity smaller than the flexural rigidity of the at least two plate-shaped portions.

10. A wearable device comprising:
a dry electrode that comprises:
an electrode core and an electrode pad electrically conductive with the electrode core;
the electrode core including a plate portion surrounded by the electrode pad, a core main body portion protruding away from the plate portion and at least partially exposed to an outside of the electrode pad, the electrode pad being comprised of conductive rubber or a conductive elastomer having a volume resistivity of 0.5 to 10.0 Ω·cm
a device main body in which is held the dry electrode; and
the device main body including a mounting portion configured to be mounted on a living body, the dry electrode being held in the device main body with the electrode pad of the dry electrode exposed so that when the mounting portion of the device main body is mounted on the living body the electrode pad of the dry electrode is brought into contact with a surface of the living body.

11. The wearable device according to claim 10, wherein at least two of the dry electrodes are held on the mounting portion of the device main body.

12. The wearable device according to claim 10, wherein four of the dry electrodes are held on the mounting portion of the device main body.

13. The wearable device according to claim 10, wherein the mounting portion of the device main body is C-shaped.

14. The wearable device according to claim 13, wherein the C-shaped mounting portion includes opposite first and second ends, the device main body including an elongated belt that is connected to the first end of the C-shaped mounting portion, the elongated belt having a free end configured to engage a latch portion on the C-shaped mounting portion to hold the wearable device on the living body.

15. The wearable device according to claim 13, wherein a first pair and a second pair of the dry electrodes are held on the mounting portion of the device main body, the C-shaped mounting portion including opposite first and second ends, the first pair of dry electrodes being positioned closer to the first end of the C-shaped mounting portion than the second pair of dry electrodes, and the second pair of dry electrodes being positioned closer to the second end of the C-shaped mounting portion than the first pair of dry electrodes.

16. The wearable device according to claim 10, further comprising a battery held in the device main body.

17. The wearable device according to claim 10, further comprising a central processing unit held in the device main body and connected to the dry electrode.

18. The wearable device according to claim 10, further comprising memory held in the device main body and storing information.

19. A method comprising:
positioning a wearable device on a living body, the wearable device comprising a device main body in which is held a dry electrode, the dry electrode comprising an electrode core and an electrode pad electrically conductive with the electrode core, the electrode core including a plate portion and a core main body portion protruding away from the plate portion, the plate portion of the electrode core being surrounded by the electrode pad, at least a part of the core main body portion that protrudes away from the plate portion being exposed outside the electrode pad, the electrode pad being comprised of conductive rubber or a conductive elastomer having a volume resistivity of 0.5 to 10.0 Ω·cm;
the positioning of the wearable device on the living body comprising positioning the wearable device so that an exposed surface of the electrode pad of the dry electrode directly contacts a surface of the living body.

20. The method according to claim 10, wherein more than one of the dry electrodes are held in the device main body, the positioning of the wearable device on the living body comprising positioning the wearable device so that an exposed surface of the electrode pad of each of the dry electrodes directly contacts a surface of the living body.
